(19) United States
(12) Patent Application Publication
(10) Pub. No.: US 2011/0144395 A1
(43) Pub. Date: Jun. 16, 2011

(54) PROCESS TO REMOVE HF FROM 1,1,1,2,2,3,4,5,5,5-DECAFLUOROPENTANE

(75) Inventors: Joan Ellen Bartelt, Wilmington, DE (US); Jeffrey P. Knapp, Wilmington, DE (US)

(73) Assignee: E.I. DU PONT DE NEMOURS AND COMPANY, WILMINGTON, DE (US)

(21) Appl. No.: 13/058,218

(22) PCT Filed: Aug. 11, 2009
(86) PCT No.: PCT/US09/53355

§ 371 Date: Feb. 9, 2011

Related U.S. Application Data
(60) Provisional application No. 61/092,924, filed on Aug. 29, 2008.

(51) Int. Cl. C07C 17/383; C07C 17/386; B01D 3/14

(52) U.S. Cl. 570/178; 203/91; 203/81; 203/94

(57) ABSTRACT

Disclosed is process for the recovery of HFC-43-10mee from a mixture comprising hydrogen fluoride and HFC-43-10mee, comprising feeding the composition comprising hydrogen fluoride and HFC-43-10mee to a distillation column, subjecting said mixture to a distillation step from which is formed a column distillate composition comprising an azeotrope or azeotrope-like composition of hydrogen fluoride and HFC-43-10mee, and a column-bottoms composition comprising HFC-43-10mee, condensing said column distillate composition to form two liquid phases, being i) an HFC-43-10mee-rich phase and ii) an HF-rich phase; and separating said two liquid phases.

### BACKGROUND INFORMATION

1. Field of the Disclosure

This disclosure relates in general to the field of purification of solvents.

2. Description of the Related Art

Lithium salts, including LiPF₆ and LiBF₄ are used as electrolytes in the manufacture of lithium ion batteries. Such batteries are finding increasing utility as power sources in a wide array of consumer electronic devices. However, the process to produce such salts includes the use of hydrogen fluoride, which is undesirable in the finished salt crystal. In addition, lithium salts such as these are inherently unstable and susceptible to hydrolysis to produce hydrogen fluoride.

### SUMMARY

Disclosed is a process for the recovery of HFC-43-10mee from a mixture comprising hydrogen fluoride and HFC-43-10mee said process comprising feeding the composition comprising hydrogen fluoride and HFC-43-10mee to a distillation column, subjecting the mixture to a distillation step from which is formed a column distillate composition comprising an azeotrope or azeotrope-like composition of hydrogen fluoride and HFC-43-10mee, and a column bottoms composition comprising HFC-43-10mee, condensing the column distillate composition to form two liquid phases, and separating the two liquid phases. One liquid phase is an HFC-43-10mee-rich phase, and the other is an HF-rich phase.

Also disclosed is a process for treating the rinsing solvent from a lithium salt purification system comprising feeding a composition comprising hydrogen fluoride and HFC-43-10mee to a distillation column removing an azeotrope or azeotrope-like composition of hydrogen fluoride and HFC-43-10mee as a distillate from the distillation column, recovering HFC-43-10mee essentially free of hydrogen fluoride from the bottom of the distillation column, condensing the azeotrope composition to form two liquid phases, being i) an HFC-43-10mee-rich phase and ii) an HF-rich phase, separating said two liquid phases, and recycling the distillation column bottoms composition to the lithium salt purification system.

Also disclosed is a process for the recovery of HFC-43-10mee from a mixture comprising hydrogen fluoride and HFC-43-10mee, comprising cooling a composition comprising hydrogen fluoride and HFC-43-10mee to a temperature low enough to form two liquid phases, being i) an HFC-43-10mee-rich phase and ii) an HF-rich phase, feeding the two liquid phases to a decanter and separating the two liquid phases, feeding the HFC-43-10mee-rich phase to a first distillation column, removing an azeotrope or azeotrope-like composition of hydrogen fluoride and HFC-43-10mee as a distillate from said first distillation column, recovering HFC-43-10mee essentially free of hydrogen fluoride from the bottom of said first distillation column; and condensing said column distillate composition and cooling said distillate composition as in the first cooling step above.

The foregoing general description and the following detailed description are exemplary and explanatory only and are not restrictive of the invention, as defined in the appended claims.

### BRIEF DESCRIPTION OF THE DRAWINGS

Embodiments are illustrated in the accompanying figures to improve understanding of concepts as presented herein.

FIG. 1 is an illustration of one embodiment of an azeotropic distillation for the separation of HF and a hydrofluorocarbon employing two distillation columns.

FIG. 2 is an illustration of one embodiment of an azeotropic distillation for the separation of HF and a hydrofluorocarbon employing one distillation column.

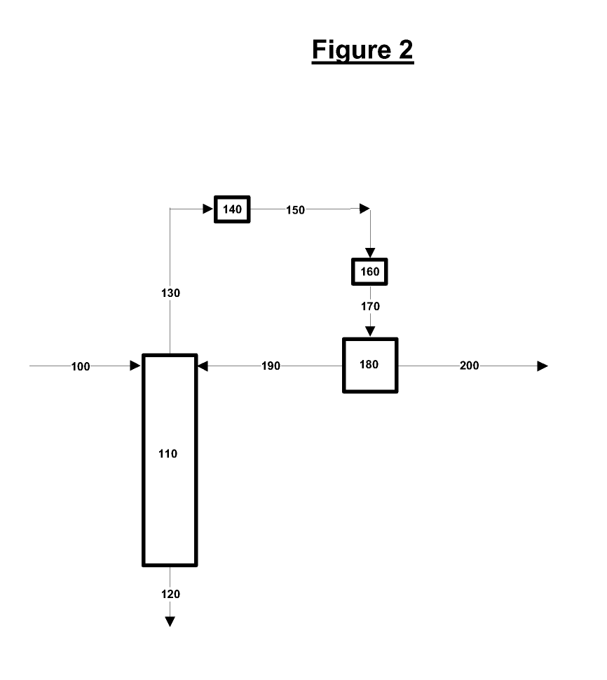

FIG. 3 is an illustration of one embodiment of an azeotropic distillation for the separation of HF and a hydrofluorocarbon wherein the mixture to be separated is first cooled and fed to a decanter.

Skilled artisans appreciate that objects in the figures are illustrated for simplicity and clarity and have not necessarily been drawn to scale. For example, the dimensions of some of the objects in the figures may be exaggerated relative to other objects to help to improve understanding of embodiments.

### DETAILED DESCRIPTION

Many aspects and embodiments have been described above and are merely exemplary and not limiting. After reading this specification, skilled artisans appreciate that other aspects and embodiments are possible without departing from the scope of the invention.

Other features and benefits of any one or more of the embodiments will be apparent from the following detailed description, and from the claims.

### DEFINITIONS AND CLARIFICATION OF TERMS

Before addressing details of embodiments described below, some terms are defined or clarified.

As used herein, an azeotropic composition is a constant boiling liquid admixture of two or more substances wherein the admixture distills without substantial composition change and behaves as a constant boiling composition. Constant boiling compositions, which are characterized as azeotropic, exhibit either a maximum or a minimum boiling point, as compared with that of the non-azeotropic mixtures of the same substances. Azeotropic compositions as used herein include homogeneous azeotropes which are liquid admixtures of two or more substances that behave as a single substance, in that the vapor, produced by partial evaporation or distillation of the liquid, has the same composition as the liquid. Azeotropic compositions as used herein also include heterogeneous azeotropes where the liquid phase splits into two or more liquid phases. In these embodiments, at the azeotropic point, the vapor phase is in equilibrium with two liquid phases and all three phases have different compositions. If the two equilibrium liquid phases of a heterogeneous azeotrope are combined and the composition of the overall liquid phase calculated, this would be identical to the composition of the vapor phase.

As used herein, the term “azeotrope-like composition” also sometimes referred to as “near azeotropic composition,” means a constant boiling, or substantially constant boiling liquid admixture of two or more substances that behaves as a single substance. One way to characterize an azeotrope-like composition is that the vapor produced by partial evaporation or distillation of the liquid has substantially the same composition as the liquid from which it was evaporated or distilled. That is, the admixture distills/refluxes without substantial composition change. Another way to characterize an azeotrope-like composition is that the bubble point vapor pressure of the composition and the dew point vapor pressure of the composition at a particular temperature are substantially the same.

Lithium salts, including LiPF₆ and LiBF₄ are used as electrolytes in the manufacture of lithium ion batteries. Such batteries are finding increasing utility as power sources in a wide array of consumer electronic devices. However, the process to produce such salts includes the use of hydrogen fluoride, which is undesirable in the finished salt crystal. In addition, lithium salts such as these are inherently unstable and susceptible to hydrolysis to produce hydrogen fluoride. The ability to remove residual hydrogen fluoride by means such as storage under vacuum, or thermal drying can be limited by the tendency of compounds such as LiPF₆ to revert to LiF and PF₅, under high vacuum or at elevated temperatures. Hydrofluorocarbon solvents have potential utility in processes to manufacture such lithium salts, including without limitation, as washing or rinsing solvent to remove residual hydrogen fluoride from the lithium salts. Generally, it is desirable to have a concentration of hydrogen fluoride of less than 50 ppm in such salts. The solvent effluent from such a process comprises a mixture of hydrofluorocarbon and hydrogen fluoride.

In consideration of processes to separate hydrogen fluoride from a hydrofluorocarbon solvent so the solvent can be reused, it has been discovered that some hydrofluorocarbons form azeotrope compositions with HF. Generally, the hydrofluorocarbon/HF azeotrope composition will boil at a lower temperature than either of the corresponding pure compounds. Several examples of such hydrofluorocarbon/HF azeotropes are disclosed in U.S. Patent Publication numbers A1.

Compositions may be formed that comprise azeotrope combinations of hydrogen fluoride with HFC-43-10mee. These include compositions comprising from about 81.8 mole percent to about 97.3 mole percent HF and from about 18.2 mole percent to about 2.7 mole percent HFC-43-10mee (which forms an azeotrope boiling at a temperature from between about −20° C. and about 100° C. and at a pressure from between about 3.0 psi (20.7 kPa) and about 198 psi (1365 kPa)).

Additionally, azeotrope-like compositions containing HF and HFC-43-10mee may be formed as well. Such azeotrope-like compositions comprise about 2.6 mole percent to about 20.1 mole percent HFC-43-10mee and about 97.4 mole percent to about 79.9 mole percent HF at temperatures ranging from about −20° C. to about 100° C. and at pressures from about 3.0 psi (20.7 kPa) and about 198 psi (1365 kPa).

It should be understood that while an azeotrope or azeotrope-like composition may exist at a particular ratio of the components at given temperatures and pressures, the azeotrope composition may also exist in compositions containing other components. These other components may include one or the other of the components of the azeotrope composition.

It has also been found that azeotrope or azeotrope-like compositions may be formed between about 3.0 psi (20.7 kPa) to about 198 psi (1365 kPa) at temperatures ranging from about −20° C. to about 100° C., said compositions consisting essentially of about 2.7 mole percent to about 18.2 mole percent HFC-43-10mee and about 97.3 mole percent to about 81.8 mole percent HF.

Compositions may be formed that consist essentially of azeotrope combinations of hydrogen fluoride with HFC-43-10mee. These include compositions consisting essentially of from about 97.3 mole percent to about 81.8 mole percent HF and from about 2.7 mole percent to about 18.2 mole percent HFC-43-10mee (which forms an azeotrope boiling at a temperature from between about −20° C. and about 100° C. and at a pressure from between about 3.0 psi (20.7 kPa) to about 198 psi (1365 kPa)).

Azeotrope-like compositions may also be formed that consist essentially of about 2.6 mole percent to about 20.1 mole percent HFC-43-10mee and about 97.4 mole percent to about 79.9 mole percent HF at temperatures ranging from about −20° C. to about 100° C. and at pressures from about 3.0 psi (20.7 kPa) to about 198 psi (1365 kPa).

At atmospheric pressure, the boiling points of hydrofluoric acid and HFC-43-10mee are about 19.5° C. and 55° C., respectively. The relative volatility at 25 psi (172 kPa) and 30° C. of HF and HFC-43-10mee was found to be nearly 1.0 as 91.9 mole percent HF and 8.1 mole percent HFC-43-10mee was approached. The relative volatility at 117 psi (807 kPa) and 80° C. was found to be nearly 1.0 as 84.8 mole percent HF and 15.2 mole percent HFC-43-10mee was approached. These data indicate that the use of conventional distillation procedures will not result in the separation of a substantially pure compound because of the low value of relative volatility of the compounds.

It has been found that azeotropes of HFC-43-10mee and HF are formed at a variety of temperatures and pressures. Azeotrope compositions may be formed between 3.0 psi (20.7 kPa) (at a temperature of −20° C.) and about 198 psi (1365 kPa) (at a temperature of 100° C.) said compositions consisting essentially of HFC-43-10mee and HF ranging from about 97.3 mole percent HF (and 2.7 mole percent HFC-43-10mee) to about 81.8 mole percent HF (and 18.2 mole percent HFC-43-10mee). An azeotrope of HF and HFC-43-10mee has been found at 30° C. and 25 psi (172 kPa) consisting essentially of about 91.9 mole percent HF and about 8.1 mole percent HFC-43-10mee. An azeotrope of HF and HFC-43-10mee has also been found at 79.8° C. and 117 psi (807 kPa) consisting essentially of about 84.8 mole percent HF and about 15.2 mole percent HFC-43-10mee. Based upon the above findings, azeotrope compositions at other temperatures and pressures may be calculated. It has been calculated that an azeotrope composition of about 97.3 mole percent HF and about 2.7 mole percent HFC-43-10mee can be formed at −20° C. and 3.0 psi (20.7 kPa) and an azeotrope composition of about 81.8 mole percent HF and about 18.2 mole percent HFC-43-10mee can be formed at 100° C. and 198 psi (1365 kPa). Accordingly, one aspect provides an azeotrope composition consisting essentially of from about 81.8 mole percent to about 97.3 mole percent HF and from about 18.2 mole percent to about 2.7 mole percent HFC-43-10mee, said composition having a boiling point of about −20° C. at 3 psi (20.7 kPa) to about 100° C. at 198 psi (1365 kPa).

It has also been found that azeotrope or azeotrope-like compositions may be formed between about 3.0 psi (20.7 kPa) to about 198 psi (1365 kPa) at temperatures ranging from about −20° C. to about 100° C., said compositions consisting essentially of about 2.6 mole percent to about 20.1 mole percent HFC-43-10mee and about 97.4 mole percent to about 79.9 mole percent HF.

It has been unexpectedly calculated that in a few cases azeotrope compositions comprising HFC-43-10-mee and HF may form two liquid phases when condensed and/or cooled. The two phases comprise a HFC-43-10mee-rich phase and an HF-rich phase. This phase behavior allows unique separation schemes utilizing liquid-liquid separation (such as decantation) of the two phases that are not possible with many saturated hydrofluorocarbons, which in general do not phase separate in the same manner.

In one embodiment, the present disclosure provides a process for separating a mixture comprising HF and HFC-43-10mee, said process comprising a) feeding the composition comprising HF and HFC-43-10mee to a distillation column; b) subjecting said mixture to a distillation step from which is formed a column distillate composition comprising an azeotrope or azeotrope-like composition of hydrogen fluoride and HFC-43-10mee, and a column-bottoms composition comprising HFC-43-10mee; c) condensing the first distillate to form two liquid phases, being i) an HF-rich phase and ii) a HFC-43-10mee-rich phase; and d) separating the two liquid phases. In another embodiment, the provided process further comprises recycling the first liquid phase enriched in HFC-43-10mee, back to the first distillation column.

In one embodiment, the distillation process to separate HFC-43-10mee and HF is a continuous process. In another embodiment, the distillation process to separate HFC-43-10mee and HF is a batch process.

Additionally, in another embodiment, the process as described above may further comprise feeding a second liquid phase, said second liquid phase being an HF-rich phase, to a second distillation zone, and recovering hydrogen fluoride as the second column bottoms composition. The second distillate composition, comprising an azeotrope or azeotrope-like composition may be recycled to the two liquid phases.

In one embodiment, after being condensed, the first distillate separates into two liquid phases upon cooling to a temperature of about 43° C. or less. In another embodiment, the first distillate is cooled to a temperature of about 30° C. or less.

In one embodiment, when the azeotropic composition of HFC-43-10mee and hydrogen fluoride has separated into two liquid phases, the HFC-43-10mee-rich phase at 30° C. is comprised of 0.5643 mole fraction HFC-43-10mee and 0.4357 mole fraction hydrogen fluoride. Expressed as weight percent, the HFC-43-10mee-rich phase is about 94.2% HFC-43-10mee, and about 5.8% HF. The HF-rich phase at 30° C. is comprised of 0.9290 mole fraction HF, and 0.0710 mole fraction HFC-43-10mee. Expressed as weight percent, the HF-rich phase is about 51% HF and about 49% HFC-43-10mee.

In another embodiment, a process is provided for separating a HFC-43-10mee from a mixture comprising hydrogen fluoride and said HFC-43-10mee, wherein said HFC-43-10mee is present in a concentration greater than the azeotrope concentration for hydrogen fluoride and said HFC-43-10mee, said process comprising: a) feeding said mixture comprising hydrogen fluoride and said HFC-43-10mee to a first distillation column; b) removing an azeotrope composition comprising hydrogen fluoride and HFC-43-10mee as a first distillate from the first distillation column; c) recovering HFC-43-10mee essentially free of hydrogen fluoride as a first bottoms composition from the first distillation column; and d) condensing the first distillate to form two liquid phases, being i) a hydrogen fluoride-rich phase and ii) a HFC-43-10mee-rich phase; d) separating the two liquid phases; and e) recycling the HFC-43-10mee-rich phase to the first distillation column.

In another embodiment, the process may further comprise: a) feeding the hydrogen fluoride-rich phase to a second distillation column, and b) recovering hydrogen fluoride essentially free of HFC-43-10mee from the bottom of the second distillation column.

In another embodiment, the second distillate comprising HF and HFC-43-10mee may be recycled to the two liquid phases.

In one embodiment, wherein the composition comprising HF and HFC-43-10mee has a concentration of HFC-43-10mee that is greater than the azeotrope concentration for HFC-43-10mee and HF, the first distillation column removes the excess HFC-43-10mee from the bottom of the column and the azeotrope composition exits the top of the column as the distillate. In another embodiment, the azeotrope composition comprising HF and HFC-43-10mee may be condensed and cooled thereby forming two liquid phases, an HF-rich phase and a HFC-43-10mee-rich phase.

In one embodiment, the HFC-43-10mee-rich phase is recycled back to the first distillation column and the HF-rich phase is fed to a second distillation column. As the HF-rich phase may have HF in excess of the azeotrope composition for HF/HFC-43-10mee, the excess HF will be removed from the second distillation column bottom.

In yet another embodiment, a process is provided for separating a HFC-43-10mee from a mixture comprising hydrogen fluoride and said HFC-43-10mee comprising a) cooling a composition comprising hydrogen fluoride and HFC-43-10mee to a temperature low enough to form two liquid phases, being i) an HFC-43-10mee-rich phase and ii) an HF-rich phase, b) feeding the two liquid phases to a decanter and separating the two liquid phases; c) feeding the HFC-43-10mee-rich phases to a first distillation column; d) removing an azeotrope or azeotrope-like composition of hydrogen fluoride and HFC-43-10mee as a distillate from said first distillation column; e) recoving HFC-43-10mee essentially free of hydrogen fluoride from the bottom of said first distillation column; and f) condensing said column distillate composition and cooling said distillate composition as in step a).

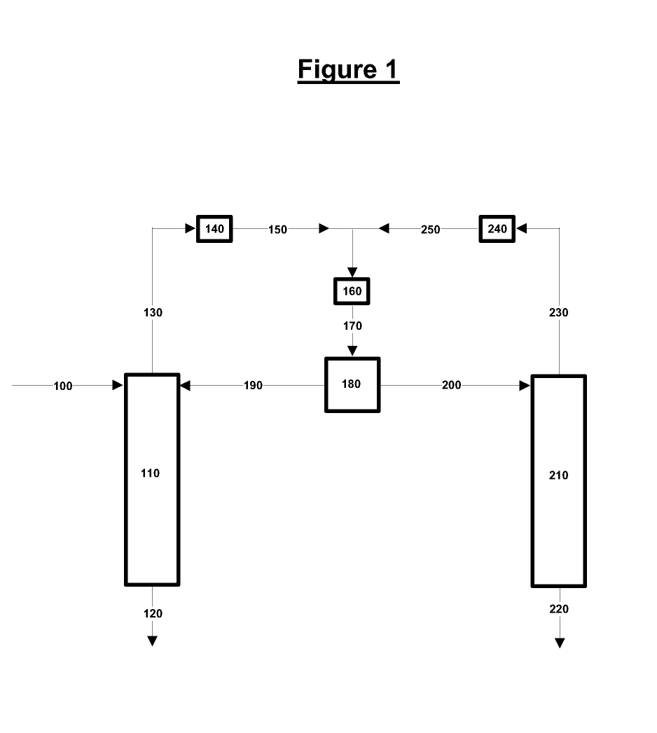

Referring now to FIG. 1, one embodiment of this process is illustrated. A composition comprising HF and HFC-43-10mee is fed to a first column 110 via stream 100. This first column is operated under appropriate conditions to approach the low-boiling HF/HFC-43-10mee azeotrope. Because HFC-43-10mee is being fed to this first column in excess of that needed to form the azeotrope with the HF, HFC-43-10mee is recovered as the bottoms of the column via stream 120, while a composition near to the HF/HFC-43-10mee azeotrope is recovered as distillate via stream 130. Stream 130 is condensed in 140, mixed with a nearly azeotropic composition recycled from a second column 210 via stream 250 and the combined stream is sub-cooled in cooler 160 and sent to decanter 180 where the combined stream 170 separates into separate HFC-43-10mee-rich (190) and HF-rich (200) streams. Stream 190 is recycled to the first column as reflux. Stream 200 is fed to the top stage of the second distillation column 210, operated under conditions to approach the HF/HFC-43-10mee azeotrope. Because the HF is being fed to this second column in excess of that needed to form the low-boiling HF/HFC-43-10mee azeotrope, HF is recovered as the bottoms of the column via stream 220 while a composition close to the HF/HFC-43-10mee azeotrope is recovered as distillate via stream 230. Stream 230 is condensed in 240, mixed with the nearly azeotropic composition from the first column via stream 150 and fed to cooler 160 and then decanter 180.

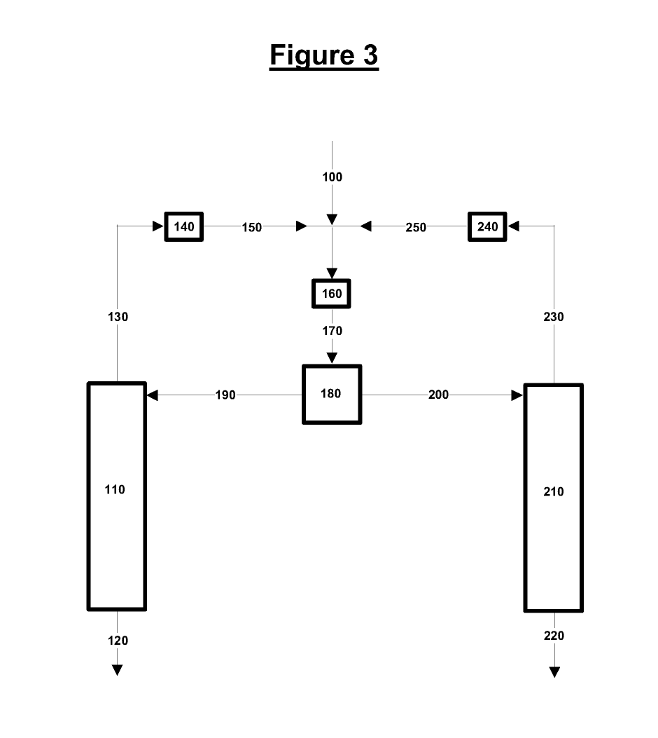

In another embodiment, the incoming feed stream comprising HF and HFC-43-10mee is fed directly to a cooler. Referring to FIG. 3, the incoming feed stream comprising HF and HFC-43-10mee is fed directly to a cooler, 160, and sub-cooled and sent to decanter 180. The combined stream 170 separates into separate HFC-43-10mee-rich (190) and HF-rich (200) streams. Stream 190 is fed to a first distillation column. This first column is operated under appropriate conditions to approach the low-boiling HF/HFC-43-10mee azeotrope. Because HFC-43-10mee is being fed to this first column in excess of that needed to form the azeotrope with the HF, HFC-43-10mee is recovered as the bottoms of the column via stream 120, while a composition near to the HF/HFC-43-10mee azeotrope is recovered as distillate via stream 130. Stream 130 is condensed in 140 and fed to the sub-cooler as stream 150. Stream 200 is fed to the top stage of the second distillation column 210, operated under conditions to approach the HF/HFC-43-10mee azeotrope. Because the HF is being fed to this second column in excess of that needed to form the low-boiling HF/HFC-43-10mee azeotrope, HF is recovered as the bottoms of the column via stream 220 while a composition close to the HF/HFC-43-10mee azeotrope is recovered as distillate via stream 230. Stream 230 is condensed in 240, and similarly fed to the sub-cooler as stream 250.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

Group numbers corresponding to columns within the Periodic Table of the elements use the “New Notation” convention as seen in the CRC Handbook of Chemistry and Physics, 81^{st} Edition (2000-2001).

Unless otherwise defined, all technical and scientific terms used herein have the same meaning as commonly understood by one of ordinary skill in the art to which this invention belongs. Although methods and materials similar or equivalent to those described herein can be used in the practice or testing of embodiments of the present invention, suitable methods and materials are described below. All publications, patent applications, patents, and other references mentioned herein are incorporated by reference in their entirety, unless a particular passage is cited. In case of conflict, the present specification, including definitions, will control. In addition, the materials, methods, and examples are illustrative only and not intended to be limiting.

### EXAMPLES

The concepts described herein will be further described in the following examples, which do not limit the scope of the invention described in the claims.

### Example 1

### Phase Studies of Mixtures of HF and HFC-43-10mee

A phase study was performed for a composition consisting essentially of HFC-43-10mee and HF, wherein the composition was varied and the vapor pressures were measured at both 30° C. and 80° C. Based upon the data from the phase studies, azeotrope compositions at other temperature and pressures have been calculated.

Table 1 provides a compilation of experimental and calculated azeotrope compositions for HF and HFC-43-10mee at specified temperatures and pressures.

TABLE 1

| | Pressure, | Mole | Mole % |
|---|---|---|---|
| Temperature, ° C. | psi (kPa) | % HF | HFC-43-10mee |
| −20 | 3.0 (20.7) | 97.3 | 2.7 |
| 0 | 7.7 (53.1) | 95.5 | 4.5 |
| 20 | 17.3 (119) | 93.2 | 6.8 |
| 30 | 25.0 (172) | 91.9 | 8.1 |
| 39.5 | 34.7 (239) | 90.7 | 9.3 |
| 40 | 35.3 (243) | 90.6 | 9.4 |
| 60 | 66.6 (459) | 87.8 | 12.2 |
| 65 | 77.3 (533) | 87.0 | 13.0 |
| 70 | 89.3 (616) | 86.3 | 13.7 |
| 75 | 103 (710) | 85.5 | 14.5 |
| 80 | 118 (814) | 84.8 | 15.2 |
| 85 | 135 (931) | 84.0 | 16.0 |
| 90 | 154 (1062) | 83.3 | 16.7 |
| 95 | 175 (1207) | 82.6 | 72.4 |
| 97.2 | 185 (1276) | 82.2 | 17.8 |
| 100 | 198 (1365) | 81.8 | 18.2 |

### Example 2

### Dew Point and Bubble Point Vapor Pressures for HFC-43-10mee

The dew point and bubble point vapor pressures for compositions disclosed herein were calculated from measured and calculated thermodynamic properties. The azeotrope-like range is indicated by the minimum and maximum concentration of HFC-43-10mee (mole percent, mol %) for which the difference in dew point and bubble point pressures is less than or equal to 3% (based upon bubble point pressure). The results are summarized in Table 2.

TABLE 2

| | | Azeotrope-like | |
|---|---|---|---|
| | | compositions, mol | |
| Temperature, | Azeotrope composition, | % HFC-43-10mee | |
| ° C. | mol % HFC-43-10mee | Minimum | Maximum |
| −20 | 2.7 | 2.6 | 2.9 |
| 0 | 4.6 | 2.8 | 4.8 |
| 20 | 6.8 | 4.9 | 7.3 |
| 70 | 13.7 | 11.2 | 15.0 |
| 100 | 18.2 | 15.0 | 20.1 |

### Example 3

This example describes a two-column azeotropic distillation process with no added entrainer for separating HFC-4310mee and HF.

A feed mixture comprising HF and HFC-4310mee is fed via stream 100 to the top stage of a first distillation column (110) containing 10 theoretical stages. In this example, the HF concentration is below the HF solubility limit in HFC-4310mee so the feed lies on the HFC-4310mee-rich side of the HF/HFC-4310mee azeotrope, which enables column 110 to be operated such that HFC-4310mee essentially free of HF is recovered from the bottom of the column as stream 120. A mixture whose composition approaches that of the HF-HFC-4310mee azeotrope is removed from the top of the column as distillate via stream 130. Distillate 130 is condensed in condenser 140, forming stream 150, combined with the condensed distillate 250 from a second distillation column and sent to cooler 160 forming sub cooled stream 170. Stream 170 is sent to decanter 180 where separate HF-rich and HFC-4310mee-rich phase fractions are formed. The HFC-4310mee-rich phase fraction is removed via stream 190 and fed to the top stage of column 110. As previously described, HFC-4310mee can be recovered from the bottom of 110 because the combined column feeds 100 and 190 have a concentration that lies on the HFC-4310mee-rich side of the azeotrope. The HF-rich phase fraction is removed from decanter 180 via stream 200 and fed to the top stage of a second distillation column 210 which contains 10 theoretical stages. Because the composition of stream 200 lies on the HF-rich side of the HF-HFC-4310mee azeotrope, column 210 may be operated such that HF, essentially free of HFC-4310mee, is recovered as the bottom product and removed via stream 220. Essentially all of the HFC-4310mee in feed 200 is removed from the top of column 210 along with enough HF to form a mixture whose composition approaches that of the HF-HFC-4310mee azeotrope, forming the distillate which is removed via stream 230. Distillate 230 is condensed in condenser 240, forming stream 250 which is combined with the condensed distillate 150 from the first distillation column as previously described.

The data in Table 3 were obtained by calculation using measured and calculated thermodynamic properties.

TABLE 3

| | Stream | | | | | |
|---|---|---|---|---|---|---|
| | | 120 | 130 | 190 | 200 | 230 |
| | 100 | C1 | C1 | 43-10 rich | HF rich | C2 |
| | Feed | bottom | distillate | liquid phase | liquid phase | distillate |
| HF mole % | 24.4 | 1.3 × 10⁻⁴ | 85.8 | 43.6 | 92.9 | 91.6 |
| HFC-43-10mee | 75.6 | 100 | 14.2 | 56.4 | 7.1 | 8.4 |
| mole % | | | | | | |
| Temp ° C. | 40 | 100 | 58.3 | 30 | 30 | 58.0 |
| Press (psi) | 64.7 | 60.7 | 59.7 | 59.2 | 59.2 | 59.7 |

### Example 4

This example demonstrates the one-column separation process to separate HFC-43-10mee from HF.

A feed mixture comprising HF and HFC-4310mee is fed via stream 100 to the top stage of distillation column (110) containing 10 theoretical stages. In this example, the HF concentration is below the HF solubility limit in HFC-4310mee so the feed lies on the HFC-4310mee-rich side of the HF/HFC-4310mee azeotrope, which enables column 110 to be operated such that HFC-4310mee essentially free of HF is recovered as the bottoms product from the column via stream 120. A mixture whose composition approaches that of the HF-HFC-4310mee azeotrope is removed from the top of the column as distillate via stream 130. Distillate 130 is condensed in condenser 140, forming stream 150, and sent to cooler 160 forming sub cooled stream 170. Stream 170 is separated into separate HF-rich and HFC-4310mee-rich phase fractions in decanter 180. The HFC-4310mee-rich phase fraction is removed from decanter 180 via stream 190 and returned to the top stage of column 110 as reflux. It is because the composition of the combined column feed streams 100 and 190 lies on the HFC-4310mee-rich side of the HF-HFC-4310mee azeotrope that essentially HF-free HFC-4310mee can be recovered from column 110. The HF-rich phase fraction is removed from decanter 180 via stream 200 and becomes the HF-rich product stream.

The data in Table 4 were obtained by calculation using measured and calculated thermodynamic properties.

TABLE 4

| | Stream | | | | |
|---|---|---|---|---|---|
| | | | | 190 | 200 |
| | 100 | 120 | 130 | 43-10 rich | HF rich |
| | Feed | C1 bottom | C1 distillate | liquid phase | liquid phase |
| HF mole | 24.4 | 1.3 × 10⁻⁴ | 85.6 | 43.6 | 92.9 |
| % | | | | | |
| HFC-43- | 75.6 | 100 | 14.4 | 56.4 | 7.1 |
| 10mee | | | | | |
| mole % | | | | | |
| Temp ° C. | 40 | 100 | 58.5 | 30 | 30 |
| Press (psi) | 64.7 | 60.7 | 59.7 | 59.2 | 59.2 |

### Example 5

This example describes a two-column azeotropic distillation process with no added entrainer for separating HFC-4310mee and HF, with the feed into the cooler and decanter.

A feed mixture comprising HF and HFC-4310mee, with the HF concentration exceeding the solubility limit at the decanter temperature [this phrase in not necessary], is mixed with the condensed distillate streams 150 and 250 from first and second distillation columns 110 and 210, respectively, and sent to cooler 160, forming subcooled stream 170. Stream 170 is separated into separate HF-rich and HFC-4310mee-rich phase fractions in decanter 180. The HFC-4310mee-rich phase fraction is removed from decanter 180 via stream 190 and fed to the top stage of a first distillation column 110 containing 10 theoretical stages and operated under conditions such that HFC-4310mee, essentially free of HF, is obtained as the column bottom product removed via stream 120. The recovery of an essentially HF-free HFC-4310mee bottoms product is possibly because the composition of stream 190 is on the HFC-4310mee-rich side of the HF-HFC-4310mee azeotrope. Essentially all of the HF in stream 190 is removed as distillate from the top of 110 via stream 130 at a composition approaching that of the HF-HFC-4310mee azeotrope. Distillate stream 130 is condensed in condenser 140, forming stream 150, and mixed with stream 100 and 250 as previously described. The HF-rich phase fraction is removed from decanter 180 via stream 200 and fed to the top stage of a second distillation column 210 which contains 10 theoretical stages. Because the composition of stream 200 lies on the HF-rich side of the HF-HFC-4310mee azeotrope, column 210 may be operated such that HF, essentially free of HFC-4310mee, is recovered as the bottom product and removed via stream 220. Essentially all of the HFC-4310mee in stream 200 is removed as distillate from the top of column 210 via stream 230 at a composition that approaches the HF-HFC-4310mee azeotrope composition. Distillate 230 is condensed in condenser 240, forming stream 250 which is combined with the condensed distillate 150 from the first distillation column and the feed stream 100 as previously described.

The data in Table 4 were obtained by calculation using measured and calculated thermodynamic properties.

TABLE 4

| | Stream | | | | | |
|---|---|---|---|---|---|---|
| | | 120 | 130 | 190 | 200 | 230 |
| | 100 | C1 | C1 | 43-10 rich | HF rich | C2 |
| | Feed | bottom | distillate | liquid phase | liquid phase | distillate |
| HF mole % | 52.3 | 1 × 10⁻⁵ | 88.2 | 43.6 | 92.9 | 91.6 |
| HFC-43-10mee | 47.7 | 100 | 11.8 | 56.4 | 7.1 | 8.4 |
| mole % | | | | | | |
| Temp ° C. | 50 | 100 | 56.5 | 30 | 30 | 58.0 |
| Press (psi) | 64.7 | 60.7 | 59.7 | 59.2 | 59.2 | 59.7 |

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

It is to be appreciated that certain features are, for clarity, described herein in the context of separate embodiments, may also be provided in combination in a single embodiment. Conversely, various features that are, for brevity, described in the context of a single embodiment, may also be provided separately or in any subcombination. Further, reference to values stated in ranges include each and every value within that range.

What is claimed is:

1. A process for the recovery of HFC-43-10mee from a mixture comprising hydrogen fluoride and HFC-43-10mee, comprising:
a.) feeding the composition comprising hydrogen fluoride and HFC-43-10mee to a distillation column;
b.) subjecting said mixture to a distillation step from which is formed a column distillate composition comprising an azeotrope or azeotrope-like composition of hydrogen fluoride and HFC-43-10mee, and a column-bottoms composition comprising HFC-43-10mee;
c.) condensing said column distillate composition to form two liquid phases, being i) an HFC-43-10mee-rich phase and ii) an HF-rich phase; and
d.) separating said two liquid phases.

2. The process of claim 1, wherein the liquid phase enriched in HFC-43-10mee is recycled to the distillation column.

3. The process of claim 1, wherein the column distillate is cooled to about 43° C. or less to produce the two liquid phases.

4. The process of claim 1, wherein the column distillate is cooled to about 30° C. or less to produce the two liquid phases.

5. The process of claim 1, further comprising feeding the HF-rich liquid phase to a second distillation column, and recovering hydrogen fluoride as a second column bottoms composition.

6. The process of claim 1, wherein said HFC-43-10mee in the composition of step a) is present in a concentration greater than the azeotrope concentration for said HFC-43-10mee and hydrogen fluoride.

7. The process of claim 6, wherein the column bottoms composition comprises HFC-43-10mee essentially free of HF.

8. The process of claim 6, wherein the column distillate is cooled to about 43° C. or less to produce the two liquid phases.

9. The process of claim 6, wherein the column distillate is cooled to about 30° C. or less to produce the two liquid phases.

10. The process of claim 6, wherein the said HFC-43-10mee-rich phase is recycled to the distillation column.

11. The process of claim 6, further comprising feeding the HF-rich liquid phase to a second distillation column, and recovering hydrogen fluoride as a second column bottoms composition.

12. A process for treating the rinsing solvent from a lithium salt purification system comprising:
a.) feeding a composition comprising hydrogen fluoride and HFC-43-10mee to a distillation column;
b.) removing an azeotrope or azeotrope-like composition of hydrogen fluoride and HFC-43-10mee as a distillate from the distillation column;
c.) recovering HFC-43-10mee essentially free of hydrogen fluoride from the bottom of the distillation column;
d.) condensing the azeotrope composition to form two liquid phases, being i) an HFC-43-10mee-rich phase and ii) an HF-rich phase;
e.) separating said two liquid phases, and
f.) recycling the distillation column bottoms composition to the lithium salt purification system.

13. The process of claim 12, wherein the column distillate is cooled to about 43° C. or less to produce the two liquid phases.

14. The process of claim 12, wherein the column distillate is cooled to about 30° C. or less to produce the two liquid phases.

15. The process of claim 12 wherein the HFC-43-10mee-rich phase of step d) is recycled to the distillation column.

16. The process of claim 12, further comprising feeding the HF-rich liquid phase to a second distillation column, and recovering hydrogen fluoride as a second column bottoms composition.

17. A process for the recovery of HFC-43-10mee from a mixture comprising hydrogen fluoride and HFC-43-10mee, comprising:
a.) cooling a composition comprising hydrogen fluoride and HFC-43-10mee to a temperature low enough to form two liquid phases, being i) an HFC-43-10mee-rich phase and ii) an HF-rich phase;
b.) feeding the two liquid phases to a decanter and separating the two liquid phases;
c.) feeding the HFC-43-10mee-rich phase to a first distillation column;
d.) removing an azeotrope or azeotrope-like composition of hydrogen fluoride and HFC-43-10mee as a distillate from said first distillation column;
e.) recovering HFC-43-10mee essentially free of hydrogen fluoride from the bottom of said first distillation column; and
f.) condensing said column distillate composition and cooling said distillate composition as in step a).

18. The process of claim 17 wherein the HF-rich phase from step b) is fed to a second distillation column, and recovering hydrogen fluoride as a second column bottoms composition.

19. The process of claim 17 wherein the composition comprising hydrogen fluoride and HFC-43-10mee is cooled to about 43° C. or less.

20. The process of claim 17 is cooled to about 30° C. or less.
